(19) United States
(12) Patent Application Publication
(10) Pub. No.: US 2012/0130242 A1
(43) Pub. Date: May 24, 2012

(54) SYSTEMS AND METHODS FOR CONCURRENTLY DISPLAYING A PLURALITY OF IMAGES USING AN INTRAVASCULAR ULTRASOUND IMAGING SYSTEM

(75) Inventors: David Burgess, San Jose, CA (US); Rick Fisher, Langhorne, PA (US); Haruko Kameda, Tokyo (JP)

(73) Assignee: Boston Scientific SciMed, Inc., Maple Grove, MN (US)

(21) Appl. No.: 13/243,255

(22) Filed: Sep. 23, 2011

Related U.S. Application Data
(60) Provisional application No. 61/417,038, filed on Nov. 24, 2010.
(60) Provisional application No. 61/484,092, filed on May 9, 2011.

(51) Int. Cl. A61B 8/14

(52) U.S. Cl. 600/443

(57) ABSTRACT

An intravascular ultrasound imaging system includes an imaging core insertable into a catheter lumen. The imaging core includes at least one transducer disposed at a distal end of a driveshaft. The at least one transducer transmits acoustic signals and transforms received echo signals to electrical signals. The at least one transducer rotates about a longitudinal axis of the catheter and moves longitudinally along a patient blood vessel as the at least one transducer transmits the acoustic signals. A control module is coupled to the imaging core. The control module includes a processor that processes received electrical signals from the at least one transducer. The processor uses the received electrical signals to generate a plurality of cross-sectional images captured along least a portion of the patient blood vessel. A display coupled to the processor concurrently displays at least two of the plurality of cross-sectional images.

### CROSS-REFERENCE TO RELATED APPLICATIONS

This application claims the benefit under 35 U.S.C. §119(e) of U.S. Provisional Patent Application Ser. No. 61/417,038 filed on Nov. 24, 2010, and U.S. Provisional Patent Application Ser. No. 61/484,092 filed on May 9, 2011, both of which are incorporated herein by reference.

### TECHNICAL FIELD

The present invention is directed to the area of imaging systems that are insertable into a patient and methods of making and using the imaging systems. The present invention is also directed to imaging systems configured and arranged to concurrently display a plurality of cross-sectional images generated along patient vasculature, as well as methods of making and using the imaging systems.

### BACKGROUND

Ultrasound devices insertable into patients have proven diagnostic capabilities for a variety of diseases and disorders. For example, intravascular ultrasound (“IVUS”) imaging systems have been used as an imaging modality for diagnosing blocked blood vessels and providing information to aid medical practitioners in selecting and placing stents and other devices to restore or increase blood flow. IVUS imaging systems have been used to diagnose atheromatous plaque build-up at particular locations within blood vessels. IVUS imaging systems can be used to determine the existence of an intravascular obstruction or stenosis, as well as the nature and degree of the obstruction or stenosis. IVUS imaging systems can be used to visualize segments of a vascular system that may be difficult to visualize using other intravascular imaging techniques, such as angiography, due to, for example, movement (e.g., a beating heart) or obstruction by one or more structures (e.g., one or more blood vessels not desired to be imaged). IVUS imaging systems can be used to monitor or assess ongoing intravascular treatments, such as angiography and stent placement in real (or almost real) time. Moreover, IVUS imaging systems can be used to monitor one or more heart chambers.

IVUS imaging systems have been developed to provide a diagnostic tool for visualizing a variety is diseases or disorders. An IVUS imaging system can include a control module (with a pulse generator, an image processor, and a monitor), a catheter, and one or more transducers disposed in the catheter. The transducer-containing catheter can be positioned in a lumen or cavity within, or in proximity to, a region to be imaged, such as a blood vessel wall or patient tissue in proximity to a blood vessel wall. The pulse generator in the control module generates electrical pulses that are delivered to the one or more transducers and transformed to acoustic pulses that are transmitted through patient tissue. Reflected pulses of the transmitted acoustic pulses are absorbed by the one or more transducers and transformed to electric pulses. The transformed electric pulses are delivered to the image processor and converted to an image displayable on the monitor.

### BRIEF SUMMARY

In one embodiment, an intravascular ultrasound imaging system includes a catheter that is insertable into a patient blood vessel. The catheter has a distal end, a proximal end, and a longitudinal axis. The catheter defines a lumen. An imaging core is configured and arranged for insertion into the lumen of the catheter. The imaging core includes at least one transducer disposed at a distal end of a driveshaft. The at least one transducer is configured and arranged for transmitting acoustic signals, receiving reflected echo signals corresponding to the transmitted acoustic signals, and transforming the received echo signals to electrical signals. The at least one transducer is configured and arranged to rotate about the longitudinal axis and move longitudinally along the patient blood vessel as the at least one transducer transmits the acoustic signals. A control module is coupled to the imaging core. The control module includes a processor configured and arranged for processing received electrical signals from the at least one transducer. The processor uses the received electrical signals to generate a plurality of cross-sectional images captured along least a portion of the patient blood vessel. A display is coupled to the processor. The display is configured and arranged to concurrently display at least two of the plurality of cross-sectional images.

In another embodiment, a computer-readable medium has processor-executable instructions that, when installed on a device, enable the device to perform actions. The actions include receiving electrical signals from at least one transducer. The electrical signals are generated from echo signals reflected from patient tissue as the at least one transducer rotates and moves longitudinally within a patient blood vessel. The received electrical signals are processed to form a plurality of cross-sectional images. Each of the plurality of cross-sectional images formed from electrical signals is received along a different longitudinal portion of the patient blood vessel such that each of the plurality of cross-sectional images is at least partially longitudinally-offset from one another. At least two of the plurality of cross-sectional images are concurrently displayed on a display coupled to the memory structure.

In yet another embodiment, an intravascular ultrasound imaging system includes a processor coupleable to at least one intravascular ultrasound transducer that is insertable into a lumen of a catheter. The at least one transducer is configured and arranged to rotate and move longitudinally along a lumen of a patient blood vessel while transmitting acoustic signals, receiving corresponding echo signals reflected from patient tissue, and transforming the received echo signals into electrical signals. The processor is configured and arranged for receiving the electrical signals from the at least one transducer and processing the received electrical signals to generate a plurality of cross-sectional images. A display is coupled to the processor and is configured and arranged for concurrently displaying at least two of the plurality of cross-sectional images.

In another embodiment, a computer-readable medium has processor-executable instructions that, when installed on a device, enable the device to perform actions. The actions include receiving electrical signals from at least one transducer. The electrical signals are generated from echo signals reflected from patient tissue as the at least one transducer rotates and moves longitudinally within a patient blood vessel. The received electrical signals are processed to form a first longitudinal image and a second longitudinal image. Each of the first longitudinal image and the second longitudinal image is formed from electrical signals received along a longitudinal portion of the patient blood vessel. A cut-plane of at least one of the first longitudinal image or the second longitudinal image is rotated until matching features can be identified along both the first longitudinal image and the second longitudinal image. One of the first longitudinal image or the second longitudinal image is superimposed over the other of the first longitudinal image or the second longitudinal image. At least one of the first longitudinal image or the second longitudinal image is translated until the matching features of the first longitudinal image and the second longitudinal image align with one another. A transformation is constructed that maps points on the first longitudinal image to corresponding points on the second longitudinal image. The first longitudinal image and the second longitudinal image are concurrently displayed on a display coupled to the device.

In yet another embodiment, an intravascular ultrasound imaging system includes a processor coupleable to at least one intravascular ultrasound transducer that is insertable into a lumen of a catheter. The at least one transducer is configured and arranged to rotate and move longitudinally along a lumen of a patient blood vessel while transmitting acoustic signals, receiving corresponding echo signals reflected from patient tissue, and transforming the received echo signals into electrical signals. The processor is configured and arranged for receiving the electrical signals from the at least one transducer and processing the received electrical signals to generate a first longitudinal image and a second longitudinal image. The first longitudinal image and the second longitudinal image are formed from electrical signals received along a longitudinal portion of the patient blood vessel. A display is coupled to the processor and is configured and arranged for concurrently displaying the first longitudinal image and the second longitudinal image.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an ultrasound imaging system suitable for insertion into a patient, the ultrasound imaging system including a catheter and a control module, according to the invention;

FIG. 2 is a schematic side view of one embodiment of the catheter of FIG. 1, according to the invention;

FIG. 3 is a schematic longitudinal cross-sectional view of one embodiment of a distal end of the catheter of FIG. 1 with an imaging core disposed in a lumen defined in the catheter, according to the invention;

FIG. 4 is a schematic view of one embodiment of a display for the ultrasound imaging system of FIG. 1, the display concurrently showing a longitudinal view of at least a portion of an imaging run and several cross-sectional images generated from different positions on the longitudinal view, according to the invention;

FIG. 5 is a schematic view of another embodiment of a display for the ultrasound imaging system of FIG. 1, the display concurrently showing a longitudinal view of at least a portion of an imaging run and several cross-sectional images generated from different positions on the longitudinal view, according to the invention;

FIG. 6 is a flow diagram of one exemplary embodiment of a technique for concurrently displaying two or more cross-sectional images generated from one or more imaging runs performed during one or more intravascular ultrasound imaging procedures, according to the invention; and

FIG. 7 is a flow diagram of one exemplary embodiment of a technique for aligning longitudinal views generated from different imaging runs performed during one or more intravascular ultrasound imaging procedures, according to the invention.

### DETAILED DESCRIPTION

The present invention is directed to the area of imaging systems that are insertable into a patient and methods of making and using the imaging systems. The present invention is also directed to imaging systems configured and arranged to concurrently display a plurality of cross-sectional images generated along patient vasculature, as well as methods of making and using the imaging systems.

The methods, systems, and devices described herein may be embodied in many different forms and should not be construed as limited to the embodiments set forth herein. Accordingly, the methods, systems, and devices described herein may take the form of an entirely hardware embodiment, an entirely software embodiment or an embodiment combining software and hardware aspects. The methods described herein can be performed using any type of computing device, such as a computer, that includes a processor or any combination of computing devices where each device performs at least part of the process.

Suitable computing devices typically include mass memory and typically include communication between devices. The mass memory illustrates a type of computer-readable media, namely computer storage media. Computer storage media may include volatile, nonvolatile, removable, non-removable, transitory, and non-transitory media implemented in any method or technology for storage of information, such as computer readable instructions, data structures, program modules, or other data. Examples of computer storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Methods of communication between devices or components of a system can include both wired and wireless (e.g., RF, optical, or infrared) communications methods and such methods provide another type of computer readable media; namely communication media.

Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

Suitable intravascular ultrasound (“IVUS”) include, but are not limited to, one or more transducers disposed on a distal end of a catheter configured and arranged for percutaneous insertion into a patient. Examples of IVUS imaging systems with catheters are found in, for example, U.S. Pat. Nos. 7,246,959; 7,306,561; and 6,945,938; as well as U.S. Patent Application Publication Nos. 2006/0100522; 2006/0106320; 2006/0173350; 2006/0253028; 2007/0016054; and 2007/0038111; all of which are incorporated herein by reference.

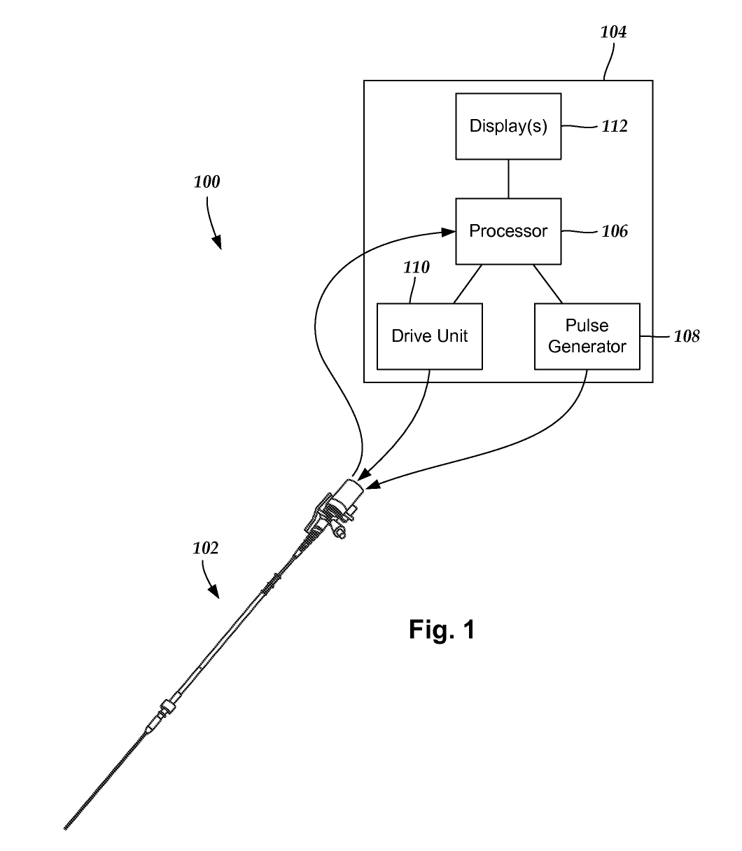

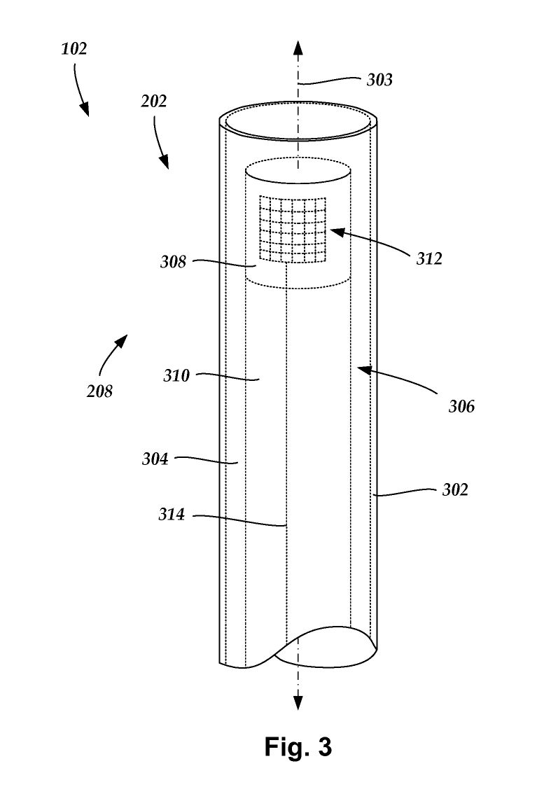

FIG. 1 illustrates schematically one embodiment of an IVUS imaging system 100. The IVUS imaging system 100 includes a catheter 102 that is coupleable to a control module 104. The control module 104 may include, for example, a processor 106, a pulse generator 108, a drive unit 110, and one or more displays 112. In at least some embodiments, the pulse generator 108 forms electric pulses that may be input to one or more transducers (312 in FIG. 3) disposed in the catheter 102.

Mechanical energy from the drive unit 110 may be used to drive an imaging core (306 in FIG. 3) disposed in the catheter 102. Electric signals transmitted from the one or more transducers (312 in FIG. 3) may be input to the processor 106 for processing. The processed electric signals from the one or more transducers (312 in FIG. 3) can be displayed as an image on the one or more displays 112. For example, a scan converter can be used to map radial scan line samples to a two-dimensional Cartesian grid to display the image on the one or more displays 112. The image can be displayed in any suitable orientation, such as cross-sectional, longitudinal, or the like.

The processor 106 may also be used to control the functioning of one or more of the other components of the control module 104. For example, the processor 106 may be used to control at least one of the frequency or duration of the electrical signals transmitted from the pulse generator 108, the rotation rate of the imaging core (306 in FIG. 3) by the drive unit 110, the velocity, duration, or length of the pullback of the imaging core (306 in FIG. 3) by the drive unit 110, or one or more properties of one or more images formed on the one or more displays 112.

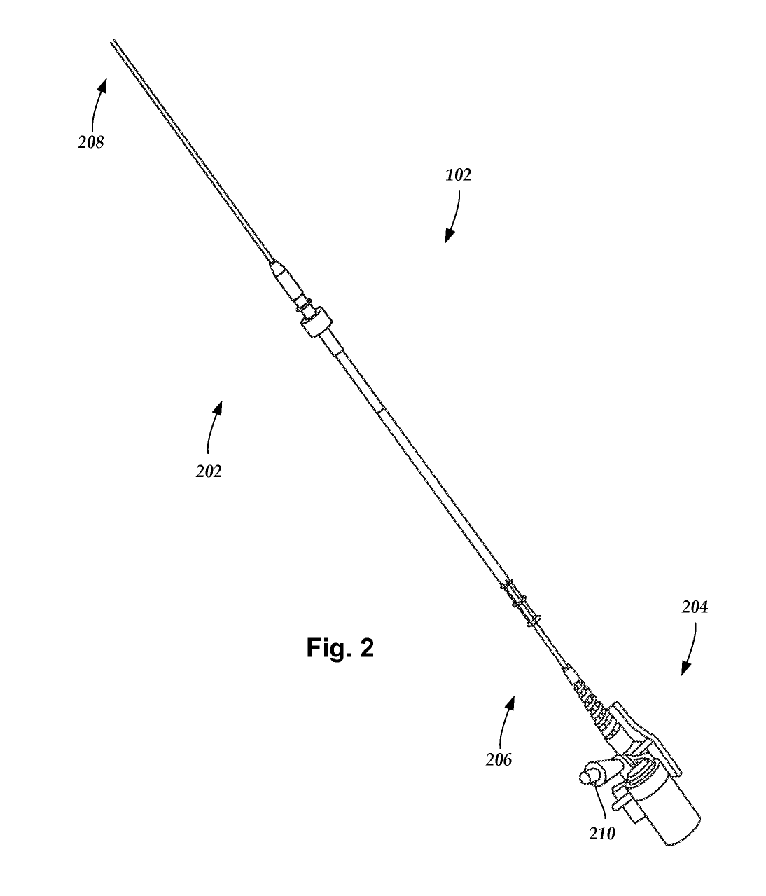

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. Optionally, the catheter 102 may define at least one flush port, such as flush port 210. The flush port 210 may be defined in the hub 204. The hub 204 may be configured and arranged to couple to the control module (104 in FIG. 1). In some embodiments, the elongated member 202 and the hub 204 are formed as a unitary body. In other embodiments, the elongated member 202 and the catheter hub 204 are formed separately and subsequently assembled together.

FIG. 3 is a schematic perspective view of one embodiment of the distal end 208 of the elongated member 202 of the catheter 102. The elongated member 202 includes a sheath 302 with a longitudinal axis 303 and a lumen 304. An imaging core 306 is disposed in the lumen 304. The imaging core 306 includes an imaging device 308 coupled to a distal end of a rotatable driveshaft 310. One or more transducers 312 may be mounted to the imaging device 308 and employed to transmit and receive acoustic signals. The sheath 302 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

In a preferred embodiment (as shown in FIG. 3), an array of transducers 312 are mounted to the imaging device 308. In alternate embodiments, a single transducer may be employed. Any suitable number of transducers 312 can be used. For example, there can be two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used. When a plurality of transducers 312 are employed, the transducers 312 can be configured into any suitable arrangement including, for example, an annular arrangement, a rectangular arrangement, or the like.

The one or more transducers 312 may be formed from one or more known materials capable of transforming applied electrical pulses to pressure distortions on the surface of the one or more transducers 312, and vice versa. Examples of suitable materials include piezoelectric ceramic materials, piezocomposite materials, piezoelectric plastics, barium titanates, lead zirconate titanates, lead metaniobates, polyvinylidenefluorides, and the like. Other transducer technologies include composite materials, single-crystal composites, and semiconductor devices (e.g., capacitive micromachined ultrasound transducers (“cMUT”), piezoelectric micromachined ultrasound transducers (“pMUT”), or the like)

The pressure distortions on the surface of the one or more transducers 312 form acoustic signals of a frequency based on the resonant frequencies of the one or more transducers 312. The resonant frequencies of the one or more transducers 312 may be affected by the size, shape, and material used to form the one or more transducers 312. The one or more transducers 312 may be formed in any shape suitable for positioning within the catheter 102 and for propagating acoustic signals of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, microfabrication, machining, and the like.

As an example, each of the one or more transducers 312 may include a layer of piezoelectric material sandwiched between a conductive acoustic lens and a conductive backing material formed from an acoustically absorbent material (e.g., an epoxy substrate with tungsten particles). During operation, the piezoelectric layer may be electrically excited by both the backing material and the acoustic lens to cause the emission of acoustic signals.

When an emitted acoustic signal with sufficient energy encounters one or more medium boundaries, such as one or more tissue boundaries, a portion of the emitted acoustic signal is reflected back to the emitting transducer as an echo signal. Each echo signal that reaches a transducer with sufficient energy to be detected is transformed to an electrical signal in the receiving transducer. The one or more transformed electrical signals are transmitted to the control module (104 in FIG. 1) where the processor 106 processes the electrical-signal characteristics to form one or more displayable images of the imaged region based, at least in part, on a collection of information from each of the acoustic signals transmitted and the echo signals received.

During an imaging procedure, the one or more transducers 312 are typically moved around in order to image a larger area than would otherwise be imaged if the one or more transducers 312 were to remain in a single position and orientation. For example, the imaging core 306 may be rotated about the longitudinal axis 303 of the catheter 102 or may be moved longitudinally along the blood vessel within which the catheter is disposed, or both. In alternate embodiments, the one or more transducers 312 are fixed in place and do not rotate. In which case, the driveshaft 310 may, instead, rotate a mirror that reflects acoustic signals between the fixed one or more transducers 312 and patient tissue.

When the one or more transducers 312 are rotated about the longitudinal axis 303 of the catheter 102 emitting acoustic signals, a cross-sectional image can be formed from electrical signals received from the one or more transducers 312. The cross-sectional image may include an entire cross-section of the region surrounding the one or more transducers 312, such as the walls of a blood vessel of interest and the tissue surrounding the blood vessel. The cross-sectional image can, optionally, be displayed on the one or more displays (112 in FIG. 1). The at least one of the imaging core 306 can be either electronically or manually rotated. In at least some embodiments, the rotation of the imaging core 306 is driven by the drive unit 110 disposed in the control module (104 in FIG. 1).

The imaging core 306 can move longitudinally along a lumen of the blood vessel within which the catheter 102 is disposed so that a plurality of at least partially longitudinally-offset cross-sectional images can be generated along a longitudinal length of the blood vessel. During an imaging procedure, the one or more transducers 312 may be refracted (i.e., pulled back) along the longitudinal length of the catheter 102 to complete an imaging run. The catheter 102 can include at least one telescoping section that can be retracted during an imaging run. In at least some embodiments, the drive unit 110 drives the pullback of the imaging core 306 during an imaging run. Alternately, pullback can be performed manually. The pullback distance of the imaging core during an imaging run can be any suitable distance including, for example, at least 5 cm, 10 cm, 15 cm, 20 cm, 25 cm, or more. The entire catheter 102 can be retracted during an imaging run either with or without the imaging core 306 moving longitudinally independently of the catheter 102. Optionally, a longitudinal view may also be generated during pullback of the one or more transducers 312 along the lumen blood vessel. The longitudinal view may include portions of one or more of the cross-sectional images.

The quality of an image produced at different depths from the one or more transducers 312 may be affected by one or more imaging properties including, for example, bandwidth, transducer focus, beam pattern, as well as the frequency of the acoustic pulse. The frequency of the acoustic signal output from the one or more transducers 312 may also affect the penetration depth of the acoustic signal output from the one or more transducers 312. In general, as the frequency of an acoustic signal is lowered, the depth of the penetration of the acoustic signal within patient tissue increases. In at least some embodiments, the IVUS imaging system 100 operates within a frequency range of 5 MHz to 100 MHz.

One or more conductors 314 can electrically couple the transducers 312 to the control module 104 (see e.g., FIG. 1). In which case, the one or more conductors 314 may extend along a longitudinal length of the rotatable driveshaft 310. In some cases, the one or more conductors 314 may extend to the control module (104 in FIG. 1).

The catheter 102 with one or more transducers 312 mounted to the distal end 208 of the imaging core 308 may be inserted percutaneously into a patient via an accessible blood vessel, such as the femoral artery, femoral vein, or jugular vein, at a site remote from the selected portion of the selected region, such as a blood vessel, to be imaged. The catheter 102 may then be advanced through the blood vessels of the patient to the selected imaging site, such as a portion of a selected blood vessel.

At least some conventional IVUS imaging systems display only a single image (e.g., cross-sectional, longitudinal, or the like) image at a time. It may, however, be useful to concurrently display a plurality of images at a time. For example, it may facilitate placement or sizing of a stent by enabling a side-by-side comparison of several images generated at two or more different positions (e.g., proximal and distal ends of one or more imaged features, landmarks, lesions, or the like) along one or more imaging runs. It may also be advantageous to display two or more cross-sectional images in combination with a longitudinal view of all, or a portion, of one or more imaging runs. In some instances, it may be advantageous to display more than one longitudinal view.

As herein described, the IVUS imaging system 100 enables a plurality of cross-sectional images to be concurrently displayed. The plurality of cross-sectional images can be generated from a single imaging run. For example, the cross-sectional images can be generated from two or more positions along the single imaging run. Alternately, the plurality of cross-sectional images can be generated from different imaging runs. For example, the cross-sectional images may be captured from the same portion of the blood vessel, yet generated at different times (e.g., before and after a given treatment, stent placement, or the like). As another example, the cross-sectional images may be captured from different imaging runs along different portions of either the same blood vessel, or a different blood vessel. In some cases, at least one of the concurrently-displayed cross-sectional images can be displayed in real-time.

Optionally, the concurrently-displayed cross-sectional images may be generated using different imaging parameters (e.g., differently-sized or differently-angled versions of the same image, different frequencies, different imaging depths, or the like). Optionally, at least one of the cross-sectional images can be moved (e.g., rotationally, translationally, or the like) or re-sized, or both, to orient the cross-sectional images with one another. When a plurality of longitudinal images are displayed, at least one of the longitudinal images can be moved (e.g., rotationally, translationally, or the like) or re-sized, or both, to align the cross-sectional images with one another.

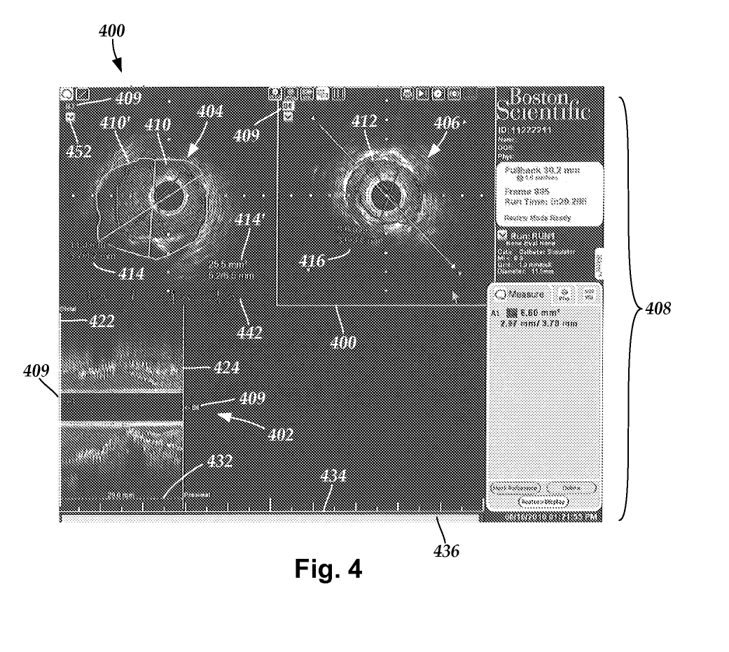

FIG. 4 is a schematic view of one embodiment of a display 400 that includes a longitudinal view 402, a first cross-sectional image 404, and a second cross-sectional image 406. The longitudinal view 402 may include all, or a portion, of one or more imaging runs. The first cross-sectional image 404 and the second cross-sectional image 406 can be cross-sectional views captured at different positions along the longitudinal view 402, or can be cross-sectional views captured along one or more different imaging runs. In FIG. 4, the longitudinal view 402, the first cross-sectional image 404, and the second cross-sectional image 406, are all captured from a single imaging run. As discussed above, the first cross-sectional image 404 and the second cross-sectional image 406 can, alternatively, be captured from different imaging runs.

It will be understood that the display 400 is an exemplary embodiment. In other embodiments, the display 400 can include additional images. For example, the display 400 can include more than two cross-sectional images, or a plurality of longitudinal views, or both. It will also be understood that the display 400 can include fewer images. For example, the display 400 may include two or more cross-sectional images, but not include any longitudinal views; or a single cross-sectional image and one or more longitudinal views.

As shown in FIG. 4, the display 400 can additionally include imaging information 408 related to at least one of the longitudinal view 402, the first cross-sectional image 404, and the second cross-sectional image 406. The imaging information 408 can include, for example, patient information, the length of pullback during an imaging run, the number of image frames generated during an imaging run, the type of catheter used during an imaging run, the date and time of an imaging run, or the like. In some cases, the display 400 can include one or more identification characters associated with one or more of the displayed image frames 409.

The display 400 can, optionally, include one or more border detection estimations (e.g., lumen borders 410, 412, or blood vessel border 410′, or both) for one or more of the cross-sectional images 404 and 406. In some instances, the display 400 can include one or more area estimations (e.g., lumen areas 414, 416, or blood vessel area 414′, or both) for one or more of the cross-sectional images 404 and 406, or the like. In at least some embodiments, one or more of the area calculations can also be included in the imaging information 408.

The longitudinal view 402 can include one or more markers 422 and 424 indicating the positioning of one or more of the first and second cross-sectional images 404 and 406, respectively, along the longitudinal view 402. Any suitable shape or symbol can be used for the markers 422, 424. In FIG. 4, the markers 422, 424 are shown as vertical lines. In FIG. 4, the markers 422 and 424 are positioned at the ends of the longitudinal view 402, indicating that the first and second cross-sectional images 404 and 406, respectively, are captured at either end of the imaging run.

In FIG. 4, two markers 422 and 424 are shown indicating the relative positioning of the cross-sectional images 404 and 406 along the longitudinal view 402. The longitudinal view 402 can include more than two markers representing cross-sectional images from more than two positions along the longitudinal view 402. In at least some embodiments, a displayed cross-sectional image can be bookmarked. Once the displayed cross-sectional image is bookmarked, a user may view other cross-sectional images, and then re-view the bookmarked cross-sectional image, as desired. In at least some embodiments, one or more bookmarked cross-sectional images can be accessible via a control 452.

Optionally, the display 400 can include one or more calculated distance measurements. The display 400 may include a measurement of the distance 432 between, for example, the first cross-sectional image 404 and the second cross-sectional image 406, the opposing ends of the longitudinal view 402, one of the cross-sectional images 404 or 406 and one of the opposing ends of the longitudinal view 402, one or more of the cross-sectional images 404 and 406 or one of more of the opposing ends of the longitudinal view 402 and a user-identified feature (e.g., a lesion, or the like) appearing on one or more of the cross-sectional images 404 and 406 or the longitudinal view 402. The distance measurement 432 can either be displayed automatically, or in response to a user request.

The display 400 can also include a measurement bar 434 positioned in proximity to the longitudinal view 402 to enable a user to estimate distances (e.g., one or more of the distance measurements discussed in the preceding paragraph) or lengths (e.g., a length of a lesion, or the like) visually observed within the longitudinal view 402 or one or more of the cross-sectional images 404, 406. The distance measurement 432 or the measurement bar 434 may be useful, for example, for facilitating determination of a stent size suitable for insertion or implantation into the patient.

In at least some embodiments, the display 400 includes one or more patient bio-signals 442 (e.g., heart rate, pulse, or the like). The patient bio-signals 442 can be either recorded or in real-time. In at least some embodiments, the imaging run is displayed in real-time. In which case, at least one of the first and second cross-sectional images 404 and 406 or the longitudinal view 402, or both, may be updated at either regular, or irregular, time intervals.

In at least some embodiments, a user can move one or more of the markers 422 and 424 to replace one or more of the currently-viewed cross-sectional images 404 and 406 with one or more other cross-sectional images corresponding to different positions along the longitudinal view 402. Alternately or additionally, the display 400 may include a scroll bar 436 that can be used by a user to scroll along the longitudinal view 402 to determine which cross-sectional images to view. Optionally, a user can add one or more cross-sectional images that can be viewed on the display 400.

In at least some embodiments, the display 400 can include two or more longitudinal views. The longitudinal views can be from imaging runs performed during the same imaging procedure, or during different imaging procedures. In some cases, the longitudinal views may include overlapping portions of the imaged blood vessel. In which case, one or more of the longitudinal views can be rotated, scaled, or scrolled along to align the overlapping portions of the blood vessel, thereby aligning the two or more longitudinal views.

In some cases, an exact alignment may not be possible (e.g., the imaging runs were performed before and after an intervention, such as before and after stent placement, or the like). In which case, it may still be possible to achieve a best fit, and thus allow a comparison of images taken from each imaging run, and the longitudinal view mappings themselves.

When aligning two longitudinal views, for example “LV1” from a first imaging run and “LV2” from a second imaging run, there are two degrees of freedom: 1) rotation of the cut plane; and 2) displacement/translation of LV1 with respect to LV2. An exemplary alignment procedure may include a medical practitioner identifying a feature (e.g., a side branch, a bifurcation, or the like) that is discernable along LV1. Identifying a discernable feature may involve rotating the cut-plane of LV1 to bring the discernable feature into view. The cut-plane of LV2 can then be rotated, if needed, to bring the matching discernable feature on LV2 into view. Either LV1 or LV2 can be superimposed over the other of LV1 or LV2. Note that the superimposed longitudinal view is preferably partially transparent, or diaphanous, so that the both LV1 and LV2 are visible when one is superimposed over the other. Either LV1 or LV2 can be translated (i.e., moved longitudinally), until the discernable features of LV1 and LV2 align with one another. In some cases, the alignment of LV1 and LV2 may be improved by further adjusting of the cut-plane of either LV1 or LV2 (or both).

It may be useful to align multiple discernable features on LV1 and LV2. In which case, once the first discernable feature is aligned, as described above, an additional discernable feature disposed on both LV1 and LV2 can be aligned through further translation of one of LV1 or LV2, while one of LV1 or LV2 is superimposed over the other of LV1 or LV2. Optionally, the cut-plane of either LV1 or LV2 can be further adjusted. Once each of the one or more discernable features is aligned, a transformation can be constructed that maps LV1 to LV2. The transformation may additionally include scaling one or more of LV1 or LV2.

In some cases, one of the first or second imaging runs may be twisted with respect to the other of the first or second imaging runs. In which case, an additional warping transformation may be needed to account for the relative twisting. A warping transformation includes superimposing either LV1 or LV2 over the other of LV1 or LV2, as discussed above, then rotating LV1 or LV2 (or both) about a central point to align LV1 and LV2. The central point of LV1 or LV2 may be initially approximated as being a center point of the catheter. Further alignment may be achieved by detecting center points in each cross-sectional image and rotating each image about that point.

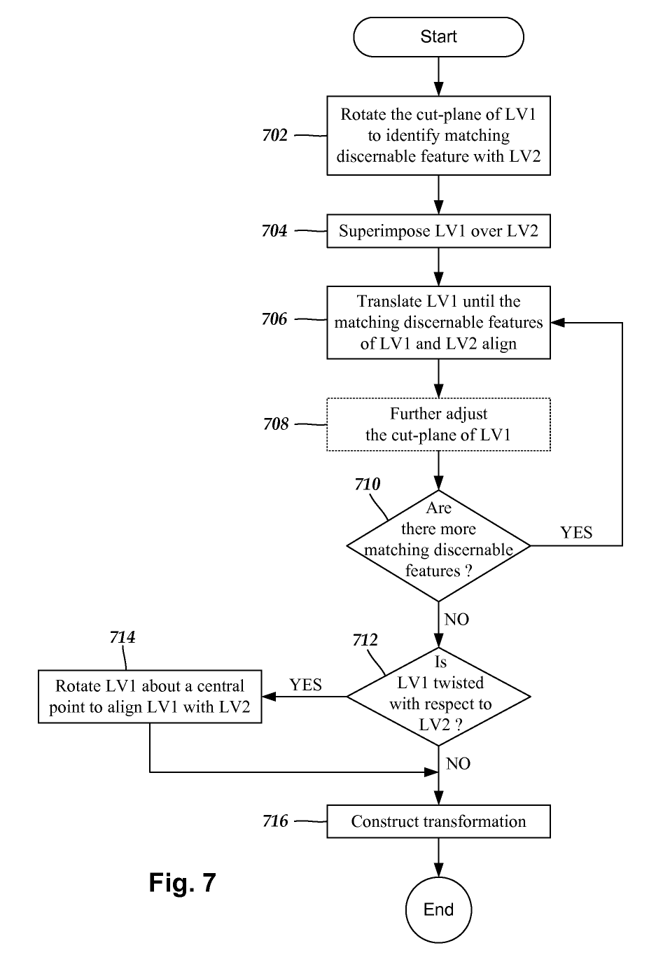

FIG. 7 is a flow diagram of one embodiment of an exemplary technique for aligning longitudinal views generated from different imaging runs performed during one or more intravascular ultrasound imaging procedures. In step 702, LV1 or LV2 is rotated along the cut-plane until matching discernable features are identified along both LV1 and LV2. In step 704, LV1 is superimposed over LV2. Alternately, LV2 is superimposed over LV1. In step 706, LV1 is translated until the matching discernable features of LV1 and LV2 align with one another. Alternately or additionally, LV2 is translated. Optionally, in step 708, the cut-plane of LV1 is further adjusted. Alternately or additionally, the cut-plane of LV2 is further adjusted. When, in step 710, an additional matching discernable feature is identified along LV1 and LV2, control is passed back to step 706. Otherwise when, in step 712, LV1 is twisted with respect to LV2, control is passed to step 714 where LV1 is rotated about a central point to align LV1 with LV2. Alternately or additionally, LV2 is rotated about a central point to align LV1 with LV2. Otherwise, in step 716, a transformation is constructed that maps LV1 to LV2.

In at least some embodiments, when the display 400 includes a plurality of cross-sectional images and a longitudinal view all formed from the same imaging run, a user of the IVUS imaging system 100 can navigate along the longitudinal view (e.g., scroll through different cross-sectional images) and perform one or more recordable actions (e.g., take distance or length measurements, make annotations, or the like).

In at least some embodiments, the display 400 can be configured to loop one or more times through a series of cross-sectional images (i.e., frames) along a selected portion of a blood vessel. For example, a user may select a frame F and a range R, where F is the center point. The display 400 can be configured and arranged to display frames from F to F+(R/2), then from F−(R/2) to F along the blood vessel. In at least some embodiments, a user can adjust the rate of looping, or the number of loops, or the like or combinations thereof. In some instances, the looping may repeat continuously until a user input is received ceasing the looping.

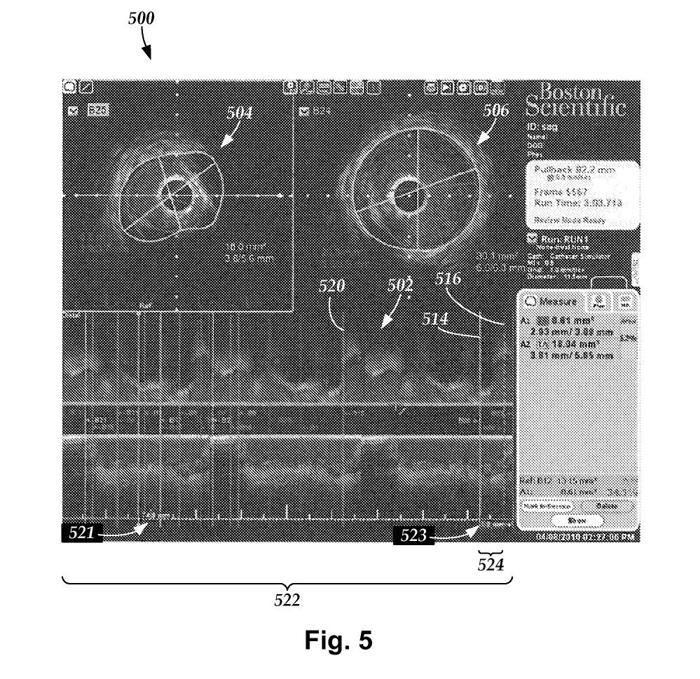

FIG. 5 is a schematic view of another embodiment of a display 500 generated by the ultrasound imaging system (100 in FIG. 1). The display 500 includes a longitudinal view 502 and two cross-sectional images 504 and 506 generated from different positions along the longitudinal view 502. The positioning of the cross-sectional images 504 and 506 along the longitudinal view 502 are represented by markers 514 and 516, respectively. As shown in FIG. 5, the longitudinal view 502 also includes additional markers, such as marker 520, representing previously-viewed and bookmarked cross-sectional images, besides the cross-sectional images 504 and 506.

As discussed above, it may facilitate stent sizing or stent placement to be able to measure the distances between two or more cross-sectional images. In FIG. 5, the display 500 shows a measurement 521 from a user-selected reference position to one or more of the displayed cross-sectional images 504 or 506. The display 500 additionally shows a measurement 523 of the distance 524 between the cross-sectional images 504 and 506.

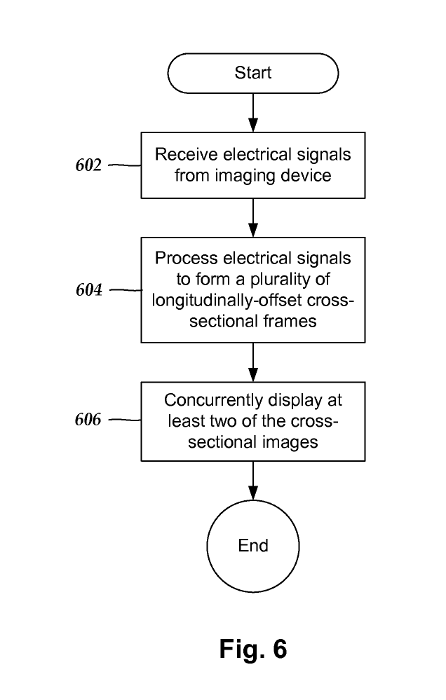

FIG. 6 is a flow diagram of one exemplary embodiment of a technique for concurrently displaying two or more cross-sectional images generated from one or more imaging runs performed during one or more intravascular ultrasound imaging procedures. In step 602, electrical signals are received from the imaging device 308 as the imaging device 308 rotates and moves longitudinally along a lumen of a patient blood vessel. In step 604, the received electrical signals are processed to form a plurality of at least partially longitudinally-offset cross-sectional images. In step 606, at least two of the plurality of cross-sectional images are concurrently displayed on the one or more displays 112. In at least some embodiments, the received electrical signals are processed to form at least one longitudinal view of a portion of the patient blood vessel that includes the plurality of cross-sectional images. Optionally, the longitudinal view is concurrently displayed along with the at least two of the plurality of cross-sectional images. In at least some embodiments, the relative positions of the displayed cross-sectional images along the longitudinal view are indicated by markers disposed over portions of the longitudinal view, or the scroll bar, or both. In at least some embodiments, the display includes the distance between the two displayed cross-sectional images. In at least some embodiments, the display includes a scroll bar for enabling a user to scroll along the longitudinal view. In at least some embodiments, one or more of the displayed cross-sectional images, as well as the position of the one or more displayed cross-sectional images on the longitudinal view, can be bookmarked. In at least some embodiments, the positions of one or more of the bookmarked cross-section images can be marked on the longitudinal view. In at least some embodiments, at least one of the at least two displayed cross-sectional images can be rotated to align a feature (e.g., a landmark, lesion, or the like) visible on one the at least two displayed cross-sectional images with the same feature appearing on another of the at least two displayed cross-sectional images. In at least some embodiments, two or more longitudinal views can be concurrently displayed. In at least some embodiments, at least one of the two or more displayed longitudinal views can be rotated, rescaled, or translated to align discernable features appearing on multiple longitudinal views.

It will be understood that each block of the flowchart illustrations, and combinations of blocks in the flowchart illustrations, as well any kind of image/signal processing, (including tissue classification), imaging transducer(s), control module, systems and methods disclosed herein, can be implemented by computer program instructions. These program instructions may be provided to a processor to produce a machine, such that the instructions, which execute on the processor, create means for implementing the actions specified in the flowchart block or blocks or described for the tissue classifier, imager, control module, systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computing device.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. An intravascular ultrasound imaging system comprising:
a catheter insertable into a patient blood vessel, the catheter having a distal end, a proximal end, and a longitudinal axis, the catheter defining a lumen;
an imaging core configured and arranged for insertion into the lumen of the catheter, the imaging core comprising at least one transducer disposed at a distal end of a driveshaft, the at least one transducer configured and arranged for transmitting acoustic signals, receiving reflected echo signals corresponding to the transmitted acoustic signals, and transforming the received echo signals to electrical signals, wherein the at least one transducer is configured and arranged to rotate about the longitudinal axis and move longitudinally along the patient blood vessel as the at least one transducer transmits the acoustic signals; and
a control module coupled to the imaging core, the control module comprising
a processor configured and arranged for processing received electrical signals from the at least one transducer, wherein the processor uses the received electrical signals to generate a plurality of cross-sectional images captured along least a portion of the patient blood vessel, and
a display coupled to the processor, wherein the display is configured and arranged to concurrently display at least two of the plurality of cross-sectional images.

2. The intravascular ultrasound imaging system of claim 1, wherein the display is configured and arranged for displaying a distance between the concurrently displayed at least two cross-sectional images.

3. The intravascular ultrasound imaging system of claim 1, wherein the processor uses the received electrical signals to generate a first longitudinal view, wherein the first longitudinal view has a first end and an opposing second end.

4. The intravascular ultrasound imaging system of claim 3, wherein the display is further configured and arranged to concurrently display the first longitudinal view with the at least two of the plurality of cross-sectional images.

5. The intravascular ultrasound imaging system of claim 4, wherein the display is further configured and arranged to display a measurement bar positioned in proximity to the first longitudinal view.

6. The intravascular ultrasound imaging system of claim 4, wherein the display is further configured and arranged to display at least one marker disposed over the first longitudinal view, the at least one marker indicating a position of one of the at least two cross-sectional images along a length of the first longitudinal view.

7. The intravascular ultrasound imaging system of claim 6, wherein the display is further configured and arranged to display a distance between the at least one marker and at least one of the first end of the first longitudinal view, the second end of the first longitudinal view, or one or more positions along the length of the first longitudinal view.

8. The intravascular ultrasound imaging system of claim 4, wherein the display is configured and arranged for displaying at least two markers disposed over the first longitudinal view, each of the at least two markers indicating the positions of a different cross-sectional image of the at least two cross-sectional images along the length of the first longitudinal view.

9. The intravascular ultrasound imaging system of claim 8, wherein the display is further configured and arranged to display a distance between the at least two markers.

10. The intravascular ultrasound imaging system of claim 3, wherein the processor uses the received electrical signals to generate a second longitudinal view.

11. The intravascular ultrasound imaging system of claim 10, wherein the first longitudinal view and the second longitudinal view contain an overlapping region, the overlapping region having at least one feature that is discernable on each of the first longitudinal view and the second longitudinal view.

12. The intravascular ultrasound imaging system of claim 1, wherein the display is further configured and arranged to concurrently display at least one patient bio-signal.

13. A method for concurrently displaying two or more cross-sectional images generated from at least one imaging run performed during an intravascular ultrasound imaging procedure, the method comprising:
providing the intravascular ultrasound imaging system of claim 1;
receiving electrical signals from the at least one transducer as the at least one transducer rotates and moves longitudinally along a lumen of a patient blood vessel;
processing the received electrical signals to form a plurality of cross-sectional images at least partially longitudinally-offset from one another along a length of the blood vessel; and
concurrently displaying at least two of the plurality of cross-sectional images on the display of the intravascular ultrasound imaging system.

14. The method of claim 13, further comprising processing the received electrical signals to form at least one longitudinal view of the length of the blood vessel along which the received electrical signals are processed to form the plurality of cross-sectional images.

15. The method of claim 14, further comprising displaying the longitudinal view concurrently with the at least two cross-sectional images.

16. The method of claim 15, wherein displaying the longitudinal view concurrently with the at least two cross-sectional images comprises marking the positions of the at least two cross-sectional images on corresponding portions of the longitudinal view.

17. The method of claim 16, further comprising displaying a distance between the marked positions of the at least two cross-sectional images.

18. The method of claim 13, further comprising replacing at least one of the at least two displayed cross-sectional images with another cross-sectional image of the plurality of cross-sectional images.

19. The method of claim 18, wherein replacing at least one of the at least two displayed cross-sectional images with another cross-sectional image of the plurality of cross-sectional images comprises bookmarking the replaced cross-sectional image and marking the position of the bookmarked cross-sectional image on the corresponding portion of the longitudinal view.

20. The method of claim 13, further comprising displaying a distance between at least one of the at least two cross-sectional images and at least one end of the longitudinal view.

21. The method of claim 13, further comprising rotating at least one of the at least two displayed cross-sectional images to orient a feature appearing on one the at least two displayed cross-sectional images with the same feature appearing on another of the at least two displayed cross-sectional images.

22. The method of claim 13, further comprising looping at least one time through at least some of the plurality of cross-sectional images captured along a selected longitudinal portion of the blood vessel.

23. A computer-readable medium having processor-executable instructions, the processor-executable instructions when installed on a device enable the device to perform actions, comprising
receiving electrical signals from at least one transducer, the electrical signals generated from echo signals reflected from patient tissue as the at least one transducer rotates and moves longitudinally within a patient blood vessel;
processing the received electrical signals to form a plurality of cross-sectional images, each of the plurality of cross-sectional images formed from electrical signals received along a different longitudinal portion of the patient blood vessel such that each of the plurality of cross-sectional images are at least partially longitudinally-offset from one another; and
concurrently displaying at least two of the plurality of cross-sectional images on a display coupled to the memory structure.

24. An intravascular ultrasound imaging system comprising:
a processor coupleable to at least one intravascular ultrasound transducer that is insertable into a lumen of a catheter, the at least one transducer configured and arranged to rotate and move longitudinally along a lumen of a patient blood vessel while transmitting acoustic signals, receiving corresponding echo signals reflected from patient tissue, and transforming the received echo signals into electrical signals, wherein the processor is configured and arranged for receiving the electrical signals from the at least one transducer and processing the received electrical signals to generate a plurality of cross-sectional images; and
a display coupled to the processor, the display configured and arranged for concurrently displaying at least two of the plurality of cross-sectional images.

25. A method for aligning longitudinal views generated from a first imaging run and a second imaging run performed during one or more intravascular ultrasound imaging procedures, the method comprising:
providing the intravascular ultrasound imaging system of claim 1;
receiving electrical signals from the at least one transducer as the at least one transducer rotates and moves longitudinally along a lumen of a patient blood vessel;
processing the received electrical signals from the first imaging run to form a first longitudinal image and from the second imaging run to form a second longitudinal image;
rotating a cut-plane of at least one of the first longitudinal image or the second longitudinal image until matching discernable features can be identified along both the first longitudinal image and the second longitudinal image;
superimposing one of the first longitudinal image or the second longitudinal image over the other of the first longitudinal image or the second longitudinal image;
translating at least one of the first longitudinal image or the second longitudinal image until the matching discernable features of the first longitudinal image and the second longitudinal image align with one another;
constructing a transformation that maps points on the first longitudinal image to corresponding points on the second longitudinal image; and
concurrently displaying the first longitudinal image and the second longitudinal image on the display of the intravascular ultrasound imaging system.

26. A computer-readable medium having processor-executable instructions, the processor-executable instructions when installed on a device enable the device to perform actions, comprising
receiving electrical signals from at least one transducer, the electrical signals generated from echo signals reflected from patient tissue as the at least one transducer rotates and moves longitudinally within a patient blood vessel;
processing the received electrical signals to form a first longitudinal image and a second longitudinal image, each of the first longitudinal image and the second longitudinal image formed from electrical signals received along a longitudinal portion of the patient blood vessel;
rotating a cut-plane of at least one of the first longitudinal image or the second longitudinal image until matching features can be identified along both the first longitudinal image and the second longitudinal image;
superimposing one of the first longitudinal image or the second longitudinal image over the other of the first longitudinal image or the second longitudinal image;
translating at least one of the first longitudinal image or the second longitudinal image until the matching features of the first longitudinal image and the second longitudinal image align with one another;
constructing a transformation that maps points on the first longitudinal image to corresponding points on the second longitudinal image; and
concurrently displaying the first longitudinal image and the second longitudinal image on a display coupled to the device.

27. An intravascular ultrasound imaging system comprising:
a processor coupleable to at least one intravascular ultrasound transducer that is insertable into a lumen of a catheter, the at least one transducer configured and arranged to rotate and move longitudinally along a lumen of a patient blood vessel while transmitting acoustic signals, receiving corresponding echo signals reflected from patient tissue, and transforming the received echo signals into electrical signals, wherein the processor is configured and arranged for receiving the electrical signals from the at least one transducer and processing the received electrical signals to generate a first longitudinal image and a second longitudinal image, the first longitudinal image and the second longitudinal image formed from electrical signals received along a longitudinal portion of the patient blood vessel; and
a display coupled to the processor, the display configured and arranged for concurrently displaying the first longitudinal image and the second longitudinal image.
